(19) United States
(12) Patent Application Publication
(10) Pub. No.: US 2005/0169854 A1
(43) Pub. Date: Aug. 4, 2005

(54) Breath scent camouflage spray

(76) Inventor: Carlos M. Coro, Coral Gables, FL (US)

Correspondence Address: GREENERG TRAURIG. P.A., 1221 Brickell Avenue, Miami, FL, 33131, US

(21) Appl. No.: 10/771,063

(22) Filed: Feb. 3, 2004

(51) Int. Cl. A61K 7/26; A61K 35/78

(52) U.S. Cl. 424/58; 424/745; 424/764; 514/185; 514/410

(57) ABSTRACT

The present invention is a breath scent camouflage spray for hunters comprising a combination of extracts of botanical herbs that are either found naturally in game environments or are consistent with plant foliage found naturally in game environments, rather than activated carbon or artificial or other masking scents that are foreign to game environments. A combination of chlorophyll, parsley and dandelion extracts is used in a preferred embodiment, with a water base solution. Glycerin is also added in a preferred embodiment. The spray has a mild pleasant scent, does not produce smut or other residues unlike activated-carbon based sprays, and camouflages human breath scent without use of chemicals that game defect as foreign to their environment.

### BACKGROUND OF THE INVENTION

A. Technical Field

The present invention relates generally to human scent hiding products and methods of use, particularly by hunters to effectively mask their breath scent from game animals. More specifically, the present invention is breath scent camouflage spray featuring a novel formulation of natural botanical herb extracts consistent with plant foliage found in game environments rather than activated carbon or artificial or other masking scents that are foreign to game environments.

B. Background

Game animals have a heightened sense of smell, often detecting predators or humans by their scent before they are detectable visually. Hunters have endeavored to mask their body scents including their breath when hunting to allow them to get closer to game and thereby improve the chances of success. Prior efforts in this field have included the use of masks worn over the hunter's face which can include activated carbon or charcoal in the fabric area that covers the hunter's mouth which absorbs and reduces the scent emitted from the mouth. U.S. Pat. Nos. 6,134,718, 6,070,578, 6,009,559 disclose clothing and masks of such types. Other odor absorbing agents used on hunting masks in the past have included chlorophyll, baking soda, activated alumina, soda lime, zeolite, calcium oxide and potassium permanganate. U.S. Pat. No. 4,503,851 discloses a face mask with a mass of absorbent material saturated with odor-masking substances. A face mask with odor absorbing molecular sieves is disclosed U.S. Pat. No. 5,269,294. Masks having odor-masking, odor-counteracting or anti-bacterial substances are also disclosed in U.S. Pat. Nos. 4,790,307 and 5,636,628. A hunting face mask with a carbon canister to control breath odor is disclosed in U.S. Pat. No. 5,117,812. U.S. Pat. No. 5,697,105, discloses a hunting mask having activated carbon to absorb breath odors. The carbon is contained in stitched together pockets between two cloth layers. The mask also contains an outer seal such as rubber or plastic to provide a tight seal, plus a one-way valve for intake of air. The absorbent may be impregnated in the fabric of the mask to provide the same function. Still other efforts have included mouthwashes that contain activated carbon, as disclosed in U.S. Pat. Nos. 6,340,447 and 6,440,415 as well as U.S. Published Patent Application Publication Nos. U.S. 2002/0197247 and U.S. 2002/0146383.

Spray products containing activated carbon for use in odor elimination for hunting are known. Activated carbon spray products, while providing reasonably effective odor elimination, have drawbacks in that the activated carbon or charcoal creates smut on the skin and mouth when applied thereto and when used as a mouthwash has an unpleasant taste, especially after multiple re-applications. Such products work by absorbing breath odors using chemicals that are generally not found in the game hunting environment, making it possible that game will still detect a foreign scent and be alerted to potential danger of nearby hunters. Thus, there remains a need for an effective product for hunters to mask their breath scents so as not to be detected by game when hunting.

The present invention addresses this need and overcomes the drawbacks of such prior efforts by providing an ingestible spray comprising a solution made up of extracts of herbs found naturally, consistent with plant foliage found in the game environment rather than substances foreign to the game environment. The present invention provides an effective camouflage of human breath which game do not detect as a foreign scent and which has a pleasant mild taste and does not produce smut, unlike activated-carbon based mouthwashes or sprays. The preferred herbal extracts include, among others, parsley, dandelion and chlorophyll and are preferably used in combination in a solution of water with glycerin.

### SUMMARY OF THE INVENTION

The present invention is a breath scent camouflage spray for use by hunters to effectively disguise their breath from game, comprising a combination of extracts of ingestible natural botanical herbs that are consistent with plant foliage in the game environment. In a preferred embodiment, the present invention is a mixture of chlorophyll extract, parsley extract and dandelion extract in water with glycerin added as a thickener and/or fixative agent. By utilizing herb extracts that are consistent with the game environment, the present invention allows hunters' breath scent to blend into the surroundings so as not to be detected by the game. The breath spray of the present invention has a pleasant mild taste and does not produce smut or other undesirable residues.

### DESCRIPTION OF A PREFERRED EMBODIMENT OF THE INVENTION

The present invention, in a preferred embodiment, comprises a combination of ingestible extracts of botanical herbs that are either present naturally in the game environment or have similar characteristics as those found in the game environment, in a water base solution. Among the preferred ingestible herbal extracts that can be used in the present invention are: chlorophyll extract, parsley extract and dandelion extract. Dandelion (taraxacum officinale) is a common plant found abundantly in most temperate zones of the northern hemisphere and is often considered a nuisance weed. Parsley (petroselinum satirum, petroselinum crispum) is an herb that is grown and found throughout the world and has been used for cooking and medicinal purposes. Chlorophyll is a green pigment in plants, often derived from alfalfa and used by humans as an internal deodorant. In a preferred embodiment a combination of chlorophyll, parsley and dandelion extracts is used, mixed in water. Glycerin is preferably also included as a thickener and/or fixative agent. The effective amount of each component used in the combination can be varied. In a preferred embodiment, the combination comprises at least 3% by volume of overall composition of glycerin, about at least 0.8% by volume of overall composition of chlorophyll extract, about at least 0.8% by volume of overall composition of parsley extract, about at least 0.8% by volume of overall composition of dandelion extract, with the balance being water. The extracts and other components are preferably combined in liquid form. The combination is created by simple mixing of the components in suitable container at room temperature. Heating or cooling is not required. In a sample formulation of the present invention in a preferred embodiment, 2 cc of glycerin was mixed with 0.5 cc of chlorophyll extract, 0.5 cc of parsley extract, 0.5 cc of dandelion extract and 60 cc of ozonated water. The resulting mixture was inserted in a spray bottle and sprayed into the mouth. The spray has a mild grass-like taste and creates a natural herbal sent that hides human breath scent. Hunters can apply and reapply the spray in desired amounts to produce, enhance or maintain the resulting breath camouflage. The spray can be used not just for hunting purposes, but also possibly for military purposes to disguise soldiers' human breath scent from guard animals or for general breath freshening purposes.

While the present invention has been shown and described herein in what are considered to be the preferred embodiments thereof, illustrating the results and advantages over the prior art obtained through the present invention, the invention is not limited to those specific embodiments. Thus, the forms of the invention shown and described herein are to be taken as illustrative and other embodiments may be selected without departing from the spirit and scope of the present invention.

1. A breath scent camouflage spray for use by hunters comprising a combination of an effective amount of one or more extracts of ingestible botanical herbs that are consistent with plant foliage that is found naturally in the game environment in a water base solution.

2. The breath scent camouflage spray of claim 1, further comprising glycerin in an effective amount.

3. The breath scent camouflage spray of claim 1, wherein said extracts of ingestible herbs are selected from the group consisting of chlorophyll, parsley and dandelion.

4. The breath scent camouflage spray of claim 1, wherein said combination of ingestible botanical herb extracts comprises about at least 2% of the total volume of said mixture.

5. A breath spray camouflage spray comprising a combination of an effective amount of chlorophyll extract, parsley extract and dandelion extract in a water base solution.

6. The breath scent camouflage spray of claim 5, further comprising glycerin in an effective amount.

7. The breath scent camouflage spray of claim 5, wherein said chlorophyll extract comprises about at least 0.8% of total volume of said mixture.

8. The breath scent camouflage spray of claim 5, wherein said parsley extract comprises about at least 0.8% of total volume of said mixture.

9. The breath scent camouflage spray of claim 5, wherein said dandelion extract comprises about at least 0.8% of total volume of said mixture.

10. The breath scent camouflage spray of claim 6, wherein said glycerin comprises about at least 3% of total volume of said mixture.

11. A breath scent camouflage spray comprising at least 3% by volume of overall composition of glycerin, about at least 0.8% by volume of overall composition of chlorophyll extract, about at least 0.8% by volume of overall composition of parsley extract, about at least 0.8% by volume of overall composition of dandelion extract, and water.
